(19) United States
(12) Patent Application Publication
(10) Pub. No.: US 2015/0320326 A1
(43) Pub. Date: Nov. 12, 2015

(54) MEDICAL VIGILANCE COVERLET

(71) Applicant: HOANA MEDICAL, INC., Honolulu, HI (US)

(72) Inventors: Patrick K. Sullivan, Kailua, HI (US); David Neil Cornejo, Kailua, HI (US)

(73) Assignee: HOANA MEDICAL, INC., Honolulu, HI (US)

(21) Appl. No.: 14/589,985

(22) Filed: Jan. 5, 2015

Related U.S. Application Data
(63) Continuation of application No. 13/791,866, filed on Mar. 8, 2013, now abandoned.
(60) Provisional application No. 61/608,564, filed on Mar. 8, 2012.

(51) Int. Cl. A61B 5/0205; A61B 5/145; A61B 5/0402; A61B 5/08; A61B 5/00; A61B 5/01

(52) U.S. Cl. CPC A61B 5/02055; A61B 5/0022; A61B 5/0024; A61B 5/002; A61B 5/01; A61B 5/0402; A61B 5/0816; A61B 5/14542

(57) ABSTRACT

A coverlet, or overlay, that contains sensors, electronics and communications systems that measures a subject's physiological condition, accommodates multiple additional add-on sensors, and transmits the data and information from the coverlet to a receiving device, which further transmits information, via wireless infrastructure, or otherwise, to data aggregators, including the Internet and Cloud.

### CROSS REFERENCE TO RELATED APPLICATIONS

This application is a continuation of U.S. application Ser. No. 13/791,866, filed Mar. 8, 2013, which claims the benefit of U.S. Provisional Application No. 61/608,564, filed Mar. 8, 2012, the contents of which are incorporated herein by reference in their entirety.

This application also incorporates by reference commonly owned U.S. application Ser. No. 09/662,006, filed Sep. 14, 2000, now U.S. Pat. No. 6,984,207, U.S. application Ser. No. 10/301,524, filed Nov. 20, 2002, now U.S. Pat. No. 7,666,151, U.S. application Ser. No. 11/004,589, filed Dec. 3, 2004, now U.S. Pat. No. 7,304,580, U.S. application Ser. No. 11/061,213, filed Feb. 18, 2005, now U.S. Pat. No. 7,652,581, U.S. application Ser. No. 11/624,200, filed Jan. 17, 2007, now U.S. Pat. No. 7,656,299, and U.S. application Ser. No. 11/624,207, filed Jan. 17, 2007, now U.S. Pat. No. 7,629,890, in their entirety.

### BACKGROUND

The present invention provides improvements that increase utilization and adoption of the LifeBed™ Medical Vigilance system currently being offered by Hoana Medical, Inc. Details concerning the existing LifeBed Medical Vigilance System are set forth on the Hoana website at www.hoana.com, which is incorporated herein by reference, and in the various Hoana patents identified above which have been incorporated herein by reference.

In the new configuration, utilizing features of the present invention, the LifeBed system is much less complex and expensive, making it available to a range of users with broad medical applications, including acute-care hospitals, sub-acute, nursing homes and the home environment. The transformation to a low cost, but connected technology, enables the Medical Vigilance Coverlet (MVC) of the present invention to provide ubiquitous healthcare information that increases transparency, while bring a community into the healthcare delivery discussion.

While especially adapted as an improvement to Hoana's LifeBed™ Medical Vigilance System, it will be appreciated that the features of the present invention can be used in other systems and applications as well.

### SUMMARY OF THE INVENTION

The MVC system is composed of three main components at the point of care (POC): (1) coverlet, (2) communication package, and a (3) display. Components outside of the POC are data aggregation, decision support, and remote connectivity that enable the full utilization of a distributed healthcare social network.

The POC components behave similar to the existing LifeBed Network Patient Vigilance System, except that there are no cables between the bed/gurney/etc. and the nurse call package.

The Medical Vigilance Coverlet (MVC) includes a coverlet or overlay, that contains sensors, electronics and communications systems to measure basic physiology without any connection to the patient. The MVC has the capacity to integrate multiple tethered and wireless physiological sensors that measure a subject's physiological condition, accommodates multiple additional add-on sensors, and transmits the data and information from the coverlet to a receiving device, which further transmits information, via wireless infrastructure, or otherwise, to data aggregators, including the Internet and Cloud.

The data aggregation and remote display server is analogous to the LifeBed Controller. In this system, however, there is provision for connecting the system to remote displays via local or wide area network connections.

Remote displays include a variety of devices, including PCs, phones, or tablets.

The features and advantages of the invention will be more readily understood from the following detailed description which should be read in conjunction with the accompanying drawings.

### BRIEF DESCRIPTION OF THE DRAWINGS

FIG. 1 is a schematic of the communications package.

FIG. 2 is a schematic of the data aggregator.

FIG. 3 is a concept schematic of a system embodying the present invention.

FIG. 4 is a block diagram of one embodiment of the point of care electronics.

FIG. 5 is a block diagram of an alternative embodiment of the point of care electronics.

FIG. 6 is a block diagram of the bedside unit and display.

FIG. 7 is a block diagram of a portable display.

FIG. 8 is a block diagram of a backend for remote monitoring and online service.

FIG. 9 is a schematic of a distributed healthcare social network embodying features of the present invention.

FIG. 10 is a graphical representation of continuous check-up and medical informatics utilizing features of the present invention.

### DETAILED DESCRIPTION OF THE PREFERRED EMBODIMENTS

Referring now in more detail to the exemplary drawings for purposes of illustrating embodiments of the invention, wherein like reference numerals designate corresponding or like elements among the several views, the system is composed of three main components at the point of care (POC): (1) coverlet, (2) communication package, and a (3) display. Components outside of the POC are data aggregation, decision support, and remote connectivity that enable the full utilization of a distributed healthcare social network.

The POC components behave similar to the existing LifeBed Network Patient Vigilance System, except that there are no cables between the bed/gurney/etc. and the nurse call package.

In a preferred embodiment, the invention includes a coverlet with embedded electronics and sensors that is made of a washable, durable fabric suitable for medical and home applications. Data is transmitted from the coverlet to a data port, configured conveniently as a wall-plug or other device, that takes data and information from the coverlet and transmits to a data aggregation portal, including the Cloud or private server, or other personal computer data aggregators. Based on the condition of the patient and risks for safety and other physiological conditions, alerts, messages and alarms are transmitted, based on the values of the data.

The data aggregation and remote display server is analogous to the LifeBed Controller. In this system, however, there is provision for connecting the system to remote displays via local or wide area network connections.

Remote displays include a variety of devices, including PCs, phones, or tablets.

Coverlet

The coverlet utilizes a suitable fabric, breathable synthetic material that can be cleaned and re-used. The coverlet contains embedded electronics, which includes the digital signal processor (DSP), signal conditioners and associated electronics that enable the coverlet embedded electronics package to be less visible and unobtrusive. The goal of the insertion and placement of the electronics package, whether included at the top of the bed, bottom foot, or side of the bed, is to be essentially invisible to the user, while transmitting via Bluetooth to a data collection point.

An advancement over prior versions is that rather than sending analog signals via a cable into a DSP board in a separate enclosure, all DSP is executed on board the bed coverlet. Information only leaves the bed as digital information. This makes the chances of interference and extraneous signals virtually non-existent, but also substantially reduces the costs.

Algorithms were re-written as fixed point DSP, versus floating point DSP, which is used where a wide dynamic range is required. Going to fixed point DSP reduced the overall costs of electronics and signal conditioning.

Sensing elements are completely enclosed

Sensing elements are located above the bed (gurney, or litter) and beneath the patient. Some amount of blankets and clothing may be between the patient and the sensors.

Sensors may completely enclose the bed (gurney, or litter) or may be pad placed on the bed and held in place with a non-slip backing.

Coverlet may be battery operated, connected to a low voltage wired power source, or powered and charged by an inductive power source. On battery power, the coverlet is capable of operating for up to 24 hrs. of constant use.

The sensors communicate with the nurse call package via Bluetooth radio, there is no data cabling. A coverlet operating under battery power will be cordless.

The coverlet electronics will also provide an interface for additional sensors connected via Bluetooth:
moisture sensor
blood pressure
body temperature
blood oxygenation level
ECG/EKG
etc.

The coverlet also contains some user interface components:
Audio output for audible alerts and voice prompts
Audio input to allow recording of voice
Alert indicator
Power on indicator
Display of current numeric readings

The coverlet will detect or report data on multiple conditions
Pulse and respiratory rate, reports current rates, alerts on high or low limit violations and alerts on percentage deviation from baseline.
Bed presence, reports or alerts on patient presence in bed
Patient movement (pressure ulcer reduction and general activity info), reports on level of movement
Pressure mapping (pressure ulcer reduction), reports values or alerts on time/pressure limit violation.
Patient sleep quality

Communications Package

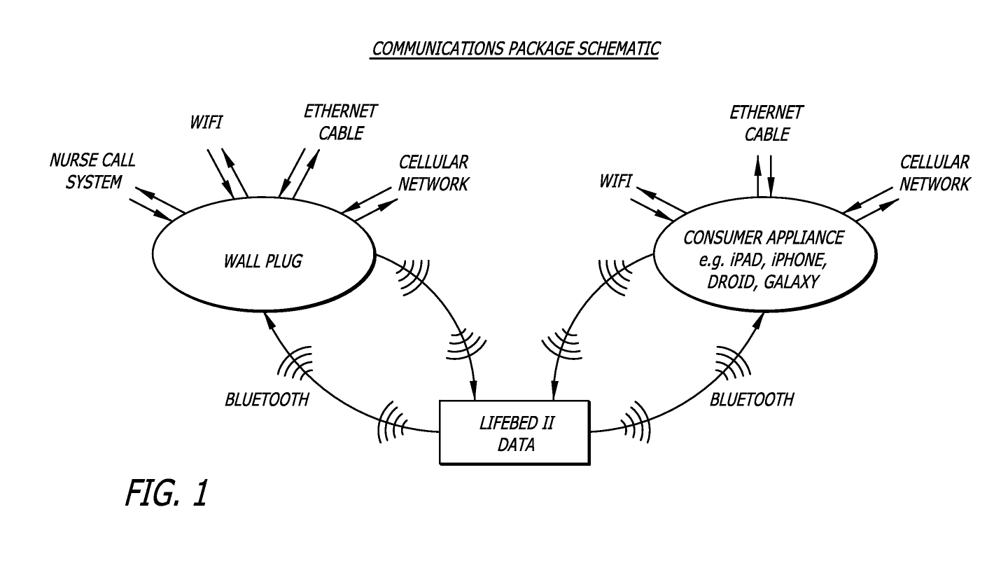

The communications package drives data and information to the data aggregator (server, Cloud, etc.), nurse-call system (acute and sub-acute care settings) or other data hosting platform. FIG. 1 shows a communications package schematic that illustrates the basic features and functions. Key features include:
Nurse-call systems typically include in-facility connectivity and cabling that accommodates an alert or signal from a patient in distress.
Small, inconspicuous package
Interfaces with standard nurse call systems (contact closure or digital)
Connects to coverlet via Bluetooth radio
Ethernet, Cellular, or WiFi, 3G, or 4G network connectivity to remote display server
LED indicators for power, connection to coverlet, and connection to remote display server
Connects to the Cloud to increase connectivity to multiple users, family, friends, physicians, nurses, etc.
Inconspicuous wall-socket that receives WiFi from the coverlet and retransmits data and information via WiFi, 3G or otherwise

Data Aggregator

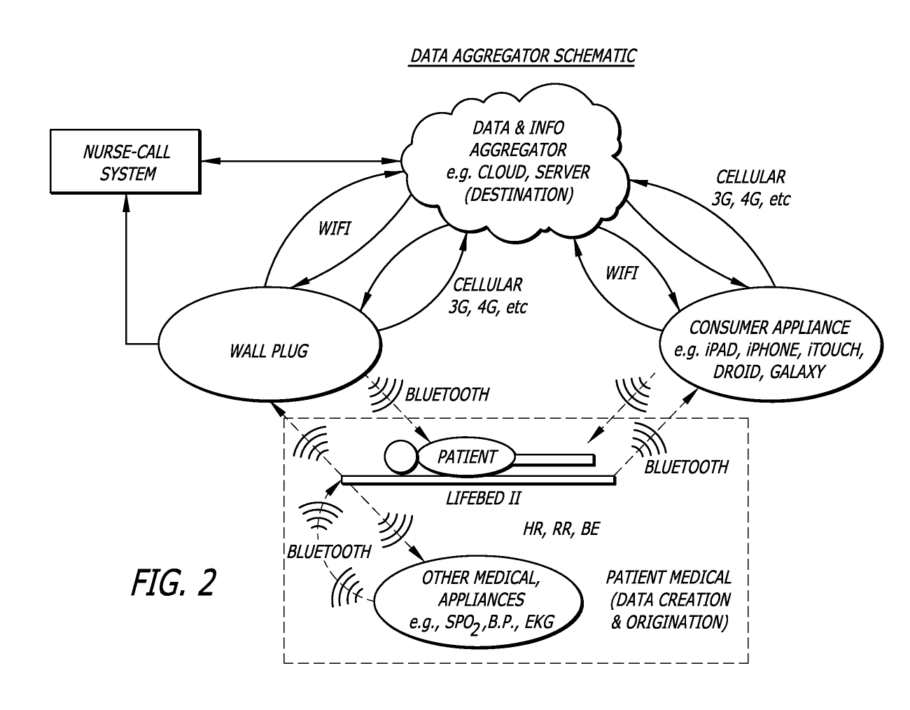

Data and information can be collected, transmitted and aggregated into any convenient platform (server, computer, Cloud) available to the user, as shown in FIG. 2 (Data Aggregator Schematic). Some users may want to rely on broadband connectivity, while others may choose to aggregate information on a convenient personal computer. The Medical Vigilance Coverlet can accommodate all these combinations:
LAN connection to nurse call package
Supports external LAN/WAN connection (intra-hospital, VPN, or Internet)
AC power
Scalable, customizable version support home, care facility, hospitals, or multiple hospitals
Serve as platform to consolidate information and alarms from multiple devices
Care Management Features:
   Identify where all the people associated with each patient are and document presence in room and timestamp, integrating simple and affordable technology such as RF ID tags that connect the caregiver with the LifeBed II system
   Identify and document which devices are in use in each patient room, using standard bar code or RF ID tag systems
   Identify and document person who turns on or off equipment, or who adjusts parameters, using caregiver identification information, e.g., ID number, barcode, or RF ID system.

Decision Support
Analyzes data from available sources
   Variability tests of data to determine possibility of adverse events
   Examine trends to identify emerging conditions and events
Draws care giver attention to likely conditions or events
Suggest steps based on protocols to further evaluate the patients' condition.
Highlights info from EHR/EMR to provide relevant information on condition to care giver
Provide access to EHR/EMR to care giver
Remote Display Server
Provides access to the system to users outside the POC.
Able to provide service via VPN or Internet
Supports multiple devices
   PCs
   Phones
   Tablets
   etc.
Formats information suitable for device
Controls access to information and configuration
Interfaces to messaging system for intra- and inter-facility communications

Display

Information derived by the Medical Vigilance Coverlet system can be displayed in many forms, aggregated into many combinations and can utilize existing displays that are convenient to the user. For example, a single bed could chose display data on a television or computer screen, or an iPhone, iPad or other computer tablet or communication appliance. Likewise, a hospital could aggregate hundreds of patients onto one display to save on costs. Once information is available via the Internet or Cloud, data can be displayed whenever and however it is convenient to the user. Examples of display feature include:
Tablet display
   Prominent display of alerts
   Ability to interactively query and display historical readings, alerts, and other events
   Displays current readings
   Allows modification of coverlet configuration
   Portable tablet with touch screen
   Battery/DC powered
   Can be wall mounted, bed mounted, or portable
   Hands free voice operation to accommodate gloved hands and lessen risk of contamination
   Access to and integrated with EHR/EMR systems
   Monitor one or more patients anywhere in system where authorized
   Provide access to decision support system
Wearable alarm indicator
   A wearable alarm indicator for nurses could reduce noise, alarm fatigue and confusion by targeting alarms only to those who need to hear them

Features of Invention

Various features are illustrated in the appended Figures. The design integrates prior Intelligent Medical Vigilance innovations, and artificial intelligence algorithms, as well as usability and user adoption centric features, including: (1) convenient; (2) easy to use; (3) target a high-risk, high-cost, and high-prevalence problems (such as falls); and (4) are either compatible with existing work patterns or have the potential for improving efficiency and time spent with patients.

Additional features that draw from an intelligent system include capabilities to address (1) adverse events, (2) injuries, (3) satisfaction, (4) competency, (5) reducing medical errors, (6) organizational outcomes such as efficiency, cost (including cost avoidance, return on investment, margins, and working capital), health care quality, and nursing retention and recruitment. As a result of integrating artificial intelligence algorithms, positive outcomes can be achieved, including: (1) improved work productivity, (2) shorter performance times, (3) improved product quality, and (4) desirable psychological and behavioral outcomes.

Alarms and warning systems used in the delivery of nursing care endeavor to detect errors before injury. A partial list includes bed exit alarms, warnings on IV pumps that signal occlusions, patient-initiated call bells, staff-initiated code alarms, wandering and elopement alarms, cardiac monitor alarms, and ventilator alarms. All of these warning systems depend on the ability of the nurse to notice the warning, process the alarm and comprehend what is happening, and finally take the appropriate action to decrease risk to the patient. In one recent study, medical/surgical nurses wanted “smart monitoring devices” that interfaced with the electronic medical record as well as with wireless communication devices. However, this strategy of using automated alarms is challenged by “alarm fatigue” stemming from the sheer number of alarms. Further, alarm fatigue is exacerbated by the well-intentioned, yet misguided decision in some existing systems to deliberately set alarms with a high false alarm rate; the effectiveness of an alerting signal drops precipitously with just a small number of false alarms.

Key Features
A wearable alarm indicator could reduce ICU noise and confusion by targeting alarms only to those who need to hear them.
Be less taxing on nurses' limited attentions
Better use the space of the room
Consolidated information and alarms from multiple devices into one location.
Integrate information about the patient's condition, devices, and the patient's body
Integrate patient health data values with trends and alarm limits
Target alarms to specific people, with an appropriate level of intrusiveness
A hands-free design in the patient room.
Barcode technology for equipment and supplies distribution and charging, automating portions of the process. Related to equipment and supply distribution, is the use of RFID technology.
Modular design to accommodate other devices
HIPAA compliant
Docking port options
Decision support algorithms and software
Integrate patient location, movement within bed, room and bathroom
Enable communications with internal other locations and departments, e.g., meal selection, entertainment, patient education.
Translation services on demand to record and transcribe essential information, which is sent to the data aggregator
Voice command activated to enable data entry and feature modification.
Nurse activated check-out to ensure that the room is made ready for the next patient

Distributed Healthcare Social Network (DHSN)

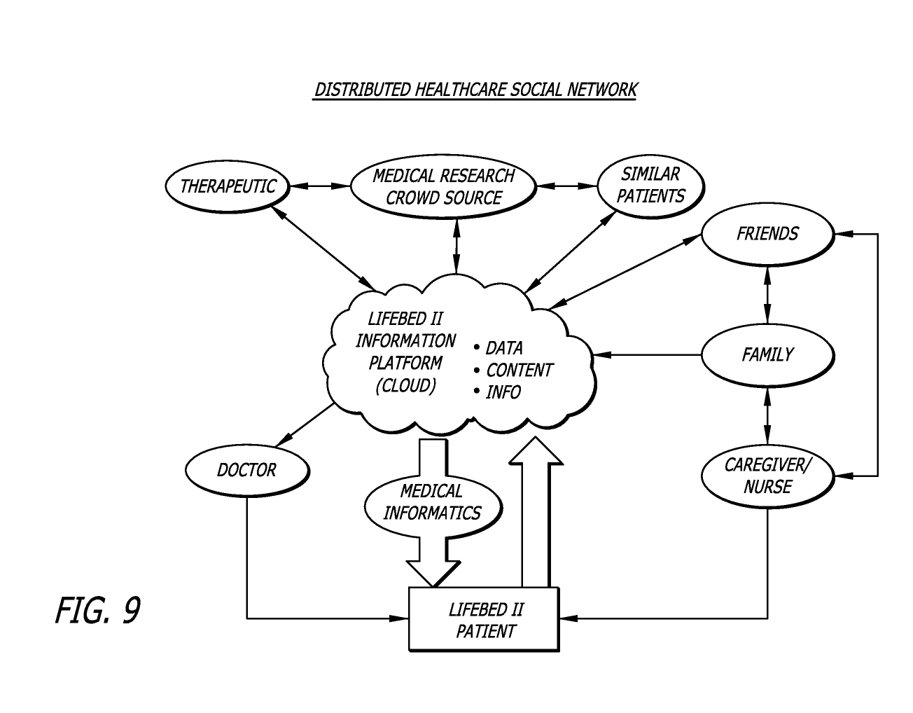

One of the keys to improving quality in hospitals is transparency, as demonstrated in a few places like Minnesota, which offers high quality care and reasonable costs paired with the most transparent hospital system in the US. To be sure, there are a variety of reasons why transparency is difficult. However, We believe that, the more transparent hospital care becomes the more accountable and fair it will be to all parties. One way to foster transparency is to connect each patient to the Internet via a Distributed Healthcare Social Network—DHSN (shown in FIG. 9). A DHSN connects together all a patient's critical “care givers”—connecting the healthcare providers, close family and friends of the patient. Although linking patients to the external community of friends and family may threaten hospitals because of the heretofore sacrosanct secrecy of what occurs within hospital walls—often protected by privacy rights legislation commonly referred to as HIPAA, (which stands for Health Insurance Portability and Accountability), We believe that accountability could be much stronger if transparency increased.

Nevertheless, even given where the law is at these days, the DHSN does not violate HIPAA because it allows the patient to choose who is included as part of their social healthcare network, and therefore, who gets access to their data. This increases the “oversight” of healthcare by those who truly care about the patient, stripping away some of the secrecy that has been tantamount in healthcare in the US. Additionally, in spite of the best intentions, today little more than lip service is typically paid to including patient family/friends in the care of the patient. The DHSN provides real information to support those individuals in contributing to the care of their loved one.

Continuous Check-Up

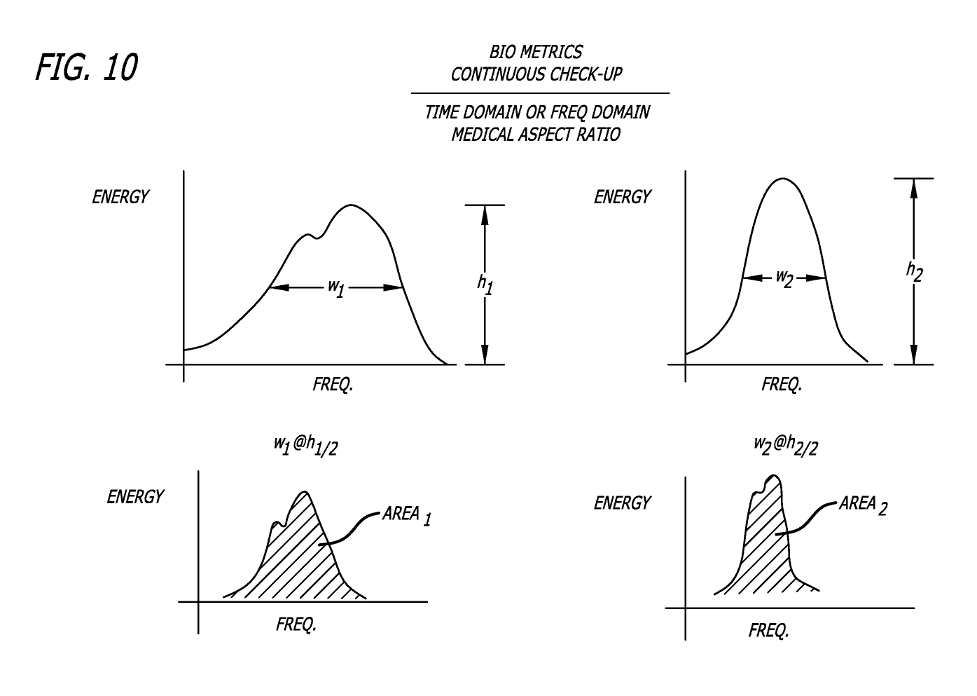

The invention described herein creates an environment for the use of unique biometrics that show subtle but important physiological changes, acquired on a regular basis when the subject/patient goes to bed at night. For example, there is a natural variability to heart and respiration rates that reflects a “healthy” condition. A significant change in this variability, as illustrated in FIG. 10 (W[1] versus w[2], or A[1] versus A[2]), can be an indicator of significant physiologic change and potential emerging medical conditions. This type of ‘continuous check-up’ is possible because of the innovations described herein, as well as the low cost and reliable connectivity. This extensive data collection and information processing creates an opportunity to continuously check-up on the patient, whereby biometrics collected can be used as a surrogate metric to identify physiologic outlier conditions that can be an earlier indicator of more complex and emerging medical conditions, whereby medical conditions of interest can be identified prior to a major medical event. Since data is collected each night the subject goes to sleep in their bed, relatively simple comparisons are possible, e.g., aspect ratio (w) of energy spectra (A), variability of heart rate and respiration rate, etc. Analysis occurs when biometrics are prepared using data collected from the coverlet, both raw data (voltages) and processed data (heart rate and respiration rate), using time series analysis techniques such as fast Fourier transforms, auto covariance, zero-up crossing, wavelets, etc.

### FIGURES

FIG. 1: Communications Package Schematic.

All data generated on the LifeBed II utilizing the present invention, including add-on medical appliance data, is produced as digital data, which is transmitted to an intermediate device or system that further transfers data and information to a data aggregator. Typically connected via Bluetooth, the communications package devices can allow for interactive communication with the LifeBed II to adjust or modify settings for patient ID, alerts, exceedance limits, etc.

FIG. 2 Data Aggregator Schematic

Data aggregation can take many forms, as long as it meets users needs and requirements. Data and information aggregated in the Cloud enables for broad access of to a multitude of users and generally increases the level of patient care transparency. However, in some acute-care settings, HIPPA and other patient confidentiality issues may require that data is aggregated in a physical plant on a secured server.

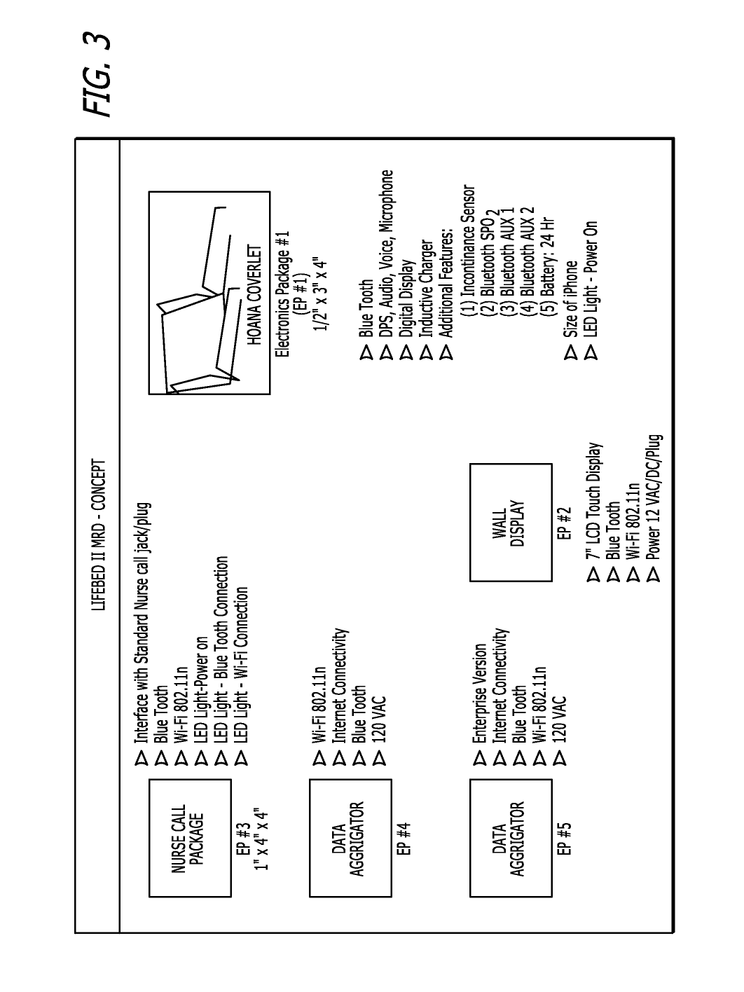

FIG. 3 is a Schematic Drawing

Concept schematic and capabilities of the LifeBed II, acute care configuration for medical surgical wards, emergency rooms, etc., using nurse-call interface in communications package.

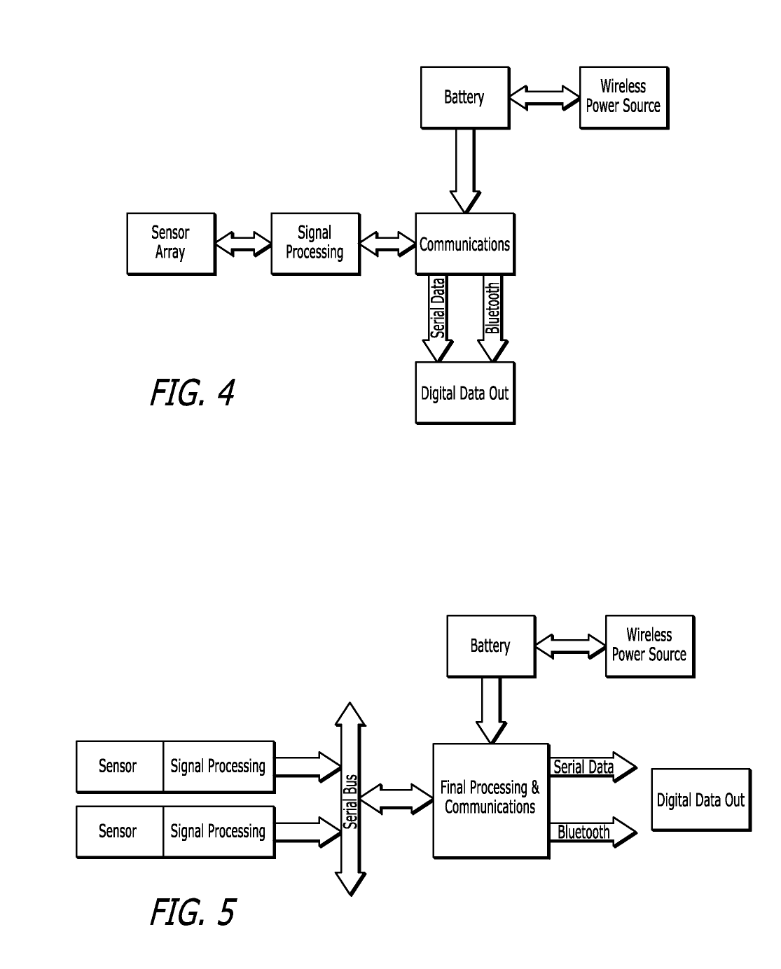

FIG. 4: Point of Care Electronics Version 1

This is the current configuration.

FIG. 5: Point of Care Electronics Version 2

This is the heavy duty/high reliability version. Innovation here is related to moving the SP to the sensor—this makes each sensor intelligent and allows better diagnosis of the sensor health. It also reduces the wiring requirements.

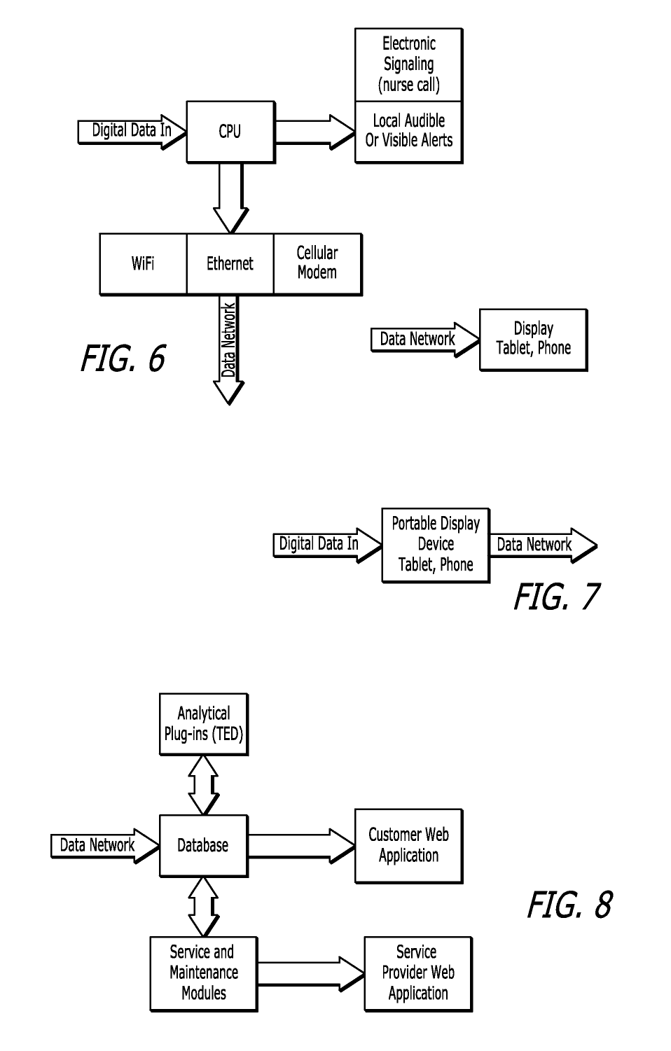

FIG. 6: Bedside Unit and Display

The module provides a hop up to a higher powered network, provides a local display, and alerts at the POC. This has two applications, in home remote monitoring and in smaller multiple bed installations.

FIG. 7: Portable Display

This enables the lowest cost and featured version. The application would generally be homes w/o remote monitoring or single bed installations.

FIG. 8: Online Service

This is the backend for remote monitoring. In consumer and small multi-bed sites this would be hosted by Hoana, in larger sites could be hosted in the customer facility.

FIG. 9: Distributed Healthcare Social Network

Utilizes an information platform, e.g., Cloud, to distribute (send and receive) patient information in an interactive way with the patients family, friends, caregivers, nurses, doctors, medical research community, etc.

FIG. 10: Continuous Check-Up, Medical Informatics

A continuous check-up can be developed with key surrogate metrics that show changes in cardiac or respiratory function, e.g., aspect ratio (w) or energy density (A), calculated from time series analysis.

The invention may be embodied in other forms without departure from the spirit and essential characteristics thereof. The embodiments described therefore are to be considered in all respects as illustrative and not restrictive. Although the present invention has been described in terms of certain preferred embodiments, other embodiments that are apparent to those of ordinary skill in the art are also within the scope of the invention. Accordingly, the scope of the invention is intended to be defined only by reference to the appended claims.

We claim:

1. A medical vigilance system comprising a coverlet with embedded sensors and electronics that collects physiological data from the patient and conveys the information to others for tracking and medical treatment.

2. The medical vigilance system of claim 1, wherein the coverlet accommodates via a Bluetooth connection a number of other physiological sensors, tethered or connected to the patient, that feed data into the coverlet.

3. The medical vigilance system of claim 1, further comprising a communications package or portal that connects the information and data from the coverlet to other locations and places, including the Internet.

4. The medical vigilance system of claim 1, wherein the coverlet has flexibility to accommodate a single user or thousands of users.

5. The medical vigilance system of claim 1, wherein the coverlet has flexibility to configure the coverlet to be used by a bed, gurney, stretcher, or other platform used to transport or support human or patient care.

6. The medical vigilance system of claim 1, further comprising crowd sourcing of data that can be utilized by social networks to determine clinically significant impacts from various therapies, remedies and other treatments of physiological conditions.

7. The medical vigilance system of claim 1, further comprising a distributed healthcare social network that brings together family, relatives, friends, physicians and nurses into the healthcare delivery of patients.

8. The medical vigilance system of claim 1, further comprising continuous check-up of data and information generated from this system.
